(19) United States
(12) Patent Application Publication
(10) Pub. No.: US 2026/0233021 A1
(43) Pub. Date: Aug. 13, 2026

(54) INVASIVE LIGHT IRRADIATION APPARATUS CAPABLE OF AUTOMATIC OUTPUT LIMITATION

(71) Applicant: WONTECH CO., LTD., Daejeon (KR)

(72) Inventors: Jong Won Kim, Seongnam (KR); Jung Hyun Kim, Seongnam (KR); Young Seok Seo, Sejong (KR); Deok Soo Kim, Daejeon (KR)

(21) Appl. No.: 19/330,872

(22) Filed: Sep. 17, 2025

(30) Foreign Application Priority Data
Feb. 11, 2025 (KR) 10-2025-0017420

(51) Int. Cl. A61N 5/06

(52) U.S. Cl. CPC A61N 5/0601; A61N 2005/0612; A61N 2005/0628; A61N 2005/063

(57) ABSTRACT

Proposed is an invasive light irradiation apparatus capable of automatic output limitation, which includes a main body configured to enable and control operation, and a needle part attachable to and detachable from the main body. The main body includes a power supply part configured to supply power, a control part configured to control operation by receiving power from the power supply part, a display part configured to output an operational status to an operator or a patient under control of the control part, a light generation part configured to generate light by receiving power from the power supply part, a light transmission part configured to transmit light generated by the light generation part, a sensor part coupled to one side of the light transmission part so as to detect reflected light, and a coupling part configured to connect the needle part to the main body.

### CROSS REFERENCE TO RELATED APPLICATION

The present application claims priority to Korean Patent Application No. 10-2025-0017420, filed February 11, 2025, the entire contents of which are incorporated herein for all purposes by this reference.

### BACKGROUND OF THE INVENTION

### FIELD OF THE INVENTION

The present disclosure relates generally to a light irradiation apparatus. More particularly, the present disclosure relates to an invasive light irradiation apparatus capable of automatic output limitation that is capable of automatically limiting output when a needle part is removed from skin.

### DESCRIPTION OF THE RELATED ART

Laser acupuncture is a non-pharmacological treatment that not only ensures safety but also, as a non-contact procedure, causes no pain or bleeding during treatment. Furthermore, since laser acupuncture uses a device that excludes physical metal needles, it eliminates concerns about infection from other diseases, so it has the great advantage of providing both comfort and relaxation to patients.

In the 1990s, it was reported that intravascular low-power laser irradiation applied had beneficial effects on neurological disorders, including ischemic cerebrovascular disease, as well as on cardiovascular disorders, urological disorders, respiratory disorders, ophthalmic disorders, diabetes, skin diseases, and arthritis.

Since then, the method of treating by intravascular irradiation with low-power lasers has been referred to as low level laser therapy (LLLT).

In the early development of laser acupuncture, a laser beam was applied to an affected area directly or by using a beam guide and a focusing lens attached to its end. However, more recently, most devices utilize optical fibers, in which a laser beam is focused into the optical fibers and delivered to the affected area without any light loss during a procedure. In particular, the advantage of this structure is that, since the laser beam is delivered into the optical fiber, an operator does not need to be close to a laser needle driving device and can naturally apply the laser beam to the affected area of a patient. However, in order to couple the laser beam emitted from the laser into the optical fiber, frequent optical alignment is required, and for this purpose, a focusing lens and optical alignment equipment must be incorporated within the system.

Acupuncture refers to a medical practice in which thin, fine needles or similar instruments are used to stimulate specific points on the human body in order to regulate the body’s qi and blood or bioenergy and to treat diseases.

The effects of acupuncture or moxibustion have been identified as involving both physical and chemical actions. Physical actions include mechanical or electrical stimulation, while chemical actions include changes in a treated area, changes in blood, hormone secretion, changes in substances in the serum, and changes in drugs.

Recently, a method called electroacupuncture (EA) has been used, in which acupuncture needles are inserted into acupuncture points and positive and negative pulsed currents are applied to the inserted needles to reduce pain and alleviate symptoms. Unlike traditional acupuncture, this method can also stimulate muscles or areas between a pair of inserted needles.

### Document of Related Art

(Patent Document 1) Korean Patent Application Publication No. 10-2021-0103811

### SUMMARY OF THE INVENTION

Accordingly, the present disclosure has been made keeping in mind the above problems occurring in the related art, and an objective of the present invention is to provide an invasive light irradiation apparatus capable of automatic output limitation, which automatically limits laser output when a needle part is invasively inserted into the skin and then removed.

In order to achieve the objectives of the present disclosure, there is provided an invasive light irradiation apparatus capable of automatic output limitation, the apparatus including: a main body configured to enable and control operation; and a needle part attachable to and detachable from the main body, wherein the main body includes: a power supply part configured to supply power; a control part configured to control operation by receiving power from the power supply part; a display part configured to output an operational status to an operator or a patient under control of the control part; a light generation part configured to generate light by receiving power from the power supply part; a light transmission part configured to transmit light generated by the light generation part; a sensor part coupled to one side of the light transmission part so as to detect reflected light; and a coupling part configured to connect the needle part to the main body, wherein the needle part includes: a second coupling member detachably coupled to the coupling part of the main body; a third light transmitter connected to one side of the second coupling member so as to transmit light generated from the main body; and an invasive device coupled to one end of the third light transmitter and having a pointed shape formed by being cut diagonally so as to be inserted into skin of a patient.

The control part may include a determination part configured to compare an intensity of light generated by the light generation part and output through the light transmission part with an intensity of light detected by the sensor part.

The light transmission part may include: a first light transmitter coupled to one side of the light generation part so as to transmit light generated by the light generation part; a second light transmitter coupled to one side of the sensor part so as to transmit reflected light to the sensor part by utilizing reflective properties of light; and a first coupling member configured to couple the first light transmitter and the second light transmitter together.

The coupling part may have one to twenty ports.

The first light transmitter and the second light transmitter may protrude separately from a first side of the first coupling member, and a coupled light transmitter may protrude from a second side of the first coupling member.

The coupled light transmitter may be coupled to the first coupling member at a first side thereof and include a coupling terminal, which is coupled to the second coupling member, at a second side thereof.

Each of the first light transmitter and the second light transmitter may be hollow to have an internal empty space, and be made of a flexible material that allows bending, with an optical fiber inserted into the internal hollow space.

The invasive device may be hollow to have an internal empty space and be made of a rigid steel material, with an optical fiber inserted into the internal empty space of the invasive device.

Light generated by the light generation part of the main body may be transmitted through optical fibers of a first light transmitter and a second light transmitter of the light transmission part, a coupled light transmitter, the third light transmitter, and the invasive device.

The sensor part may be connected to one side of a second light transmitter, and the second light transmitter may detect a portion of light that is reflected back when the light generated by the main body is emitted through the needle part.

The determination part may compare an intensity of reflected light detected by the sensor part with an intensity of light generated by the main body and emitted to the needle part so as to determine whether the needle part is invasively inserted.

When the control part detects an operation signal as light energy P1, the light generation part may generate and output light, and the sensor part senses reflected light for a first period of a seconds, wherein the P1 may be set within a range from 5mW to 50mW, and the a seconds may be set within a range from 0.1 seconds to 5.0 seconds.

When the needle part is invasively inserted into the skin, light may be continuously output for N minutes set by the control part, wherein the N minutes may be set within a range from 2 minutes to 30 minutes.

When the needle part is invasively inserted into the skin, light is continuously output for the N minutes set by the control part, and, simultaneously, the sensor part may sense reflected light for a second period of b seconds, wherein the b seconds may be set within a range from 0.1 seconds to 5.0 seconds.

According to these features, when the needle part capable of light irradiation is invasively inserted under the skin to directly illuminate light under the skin, and the needle part is removed after the procedure, light output can be automatically limited, so that it is possible to prevent accidents that may occur when the light is exposed to the air and scattered and the eyes or skin of an operator or a patient are directly irradiated with the light.

In addition, it is possible to prevent accidents that may occur when the eyes or skin of an operator or a patient are directly irradiated with light due to the exposure and scattering of the light to the air in a case in which the light continues to be emitted when the needle part is removed from the skin due to the movement of the operator or the patient.

### BRIEF DESCRIPTION OF THE DRAWINGS

The above and other objectives, features, and other advantages of the present disclosure will be more clearly understood from the following detailed description when taken in conjunction with the accompanying drawings, in which:

FIG. 1 is a configuration diagram of an invasive light irradiation apparatus capable of automatic output limitation according to an embodiment of the present disclosure;

FIG. 2 is a schematic view of a needle part of the invasive light irradiation apparatus capable of automatic output limitation according to the embodiment of the present disclosure;

FIG. 3 is a schematic view of the invasive light irradiation apparatus capable of automatic output limitation according to the embodiment of the present disclosure; and

FIG. 4 is a graph showing an irradiation cycle of the invasive light irradiation apparatus capable of automatic output limitation according to the embodiment of the present disclosure.

### DETAILED DESCRIPTION OF THE INVENTION

Hereinafter, an embodiment of the present disclosure will be described in detail with reference to the accompanying drawings so that those skilled in the art to which the present disclosure pertains can easily implement it. However, the present disclosure is not limited to the embodiment described herein and may be implemented in various other forms. In addition, in the drawings, portions unrelated to the present disclosure have been omitted in order to clearly describe the present disclosure, and similar reference numerals are used throughout the specification for similar portions.

Throughout the present specification, when a certain part is described as being “connected (coupled, contacted, or joined)” to another part, it is intended to include not only cases where the two parts are directly connected, but also cases where the two parts are indirectly connected with another member placed therebetween. In addition, when a certain part is described as “including” a specific component, it is to be understood, unless expressly stated otherwise, that the part does not exclude the presence of other components but may further include additional components.

Terms used in the present specification are employed merely to describe a specific embodiment and are not intended to limit the present disclosure. Unless clearly indicated otherwise by the context, singular expressions also include plural expressions. In the present specification, terms such as “comprise” or “have” are intended to specify the presence of a feature, a number, a step, an operation, a component, a part, or combination thereof, but should not be construed as precluding the possibility of the presence or addition of one or more other features, numbers, steps, operations, components, parts, or combinations thereof.

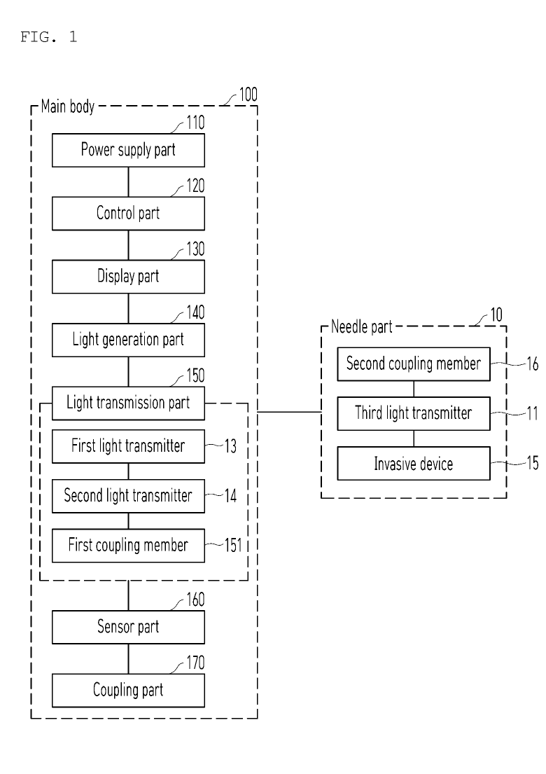

An invasive light irradiation apparatus capable of automatic output limitation according to an embodiment of the present disclosure will be described with reference to FIGS. 1 to 4.

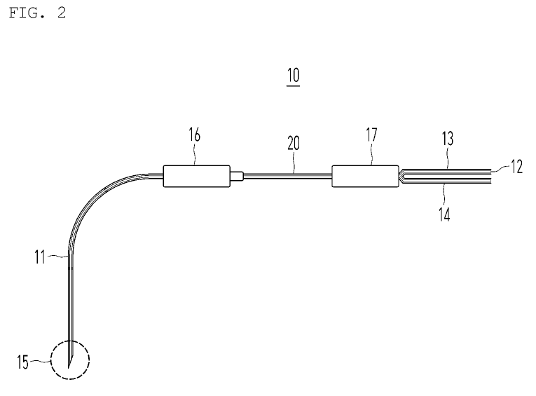

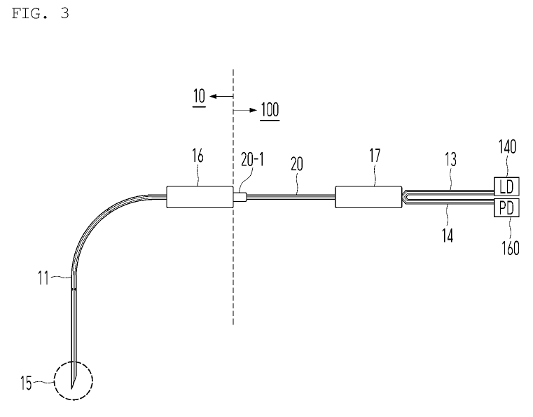

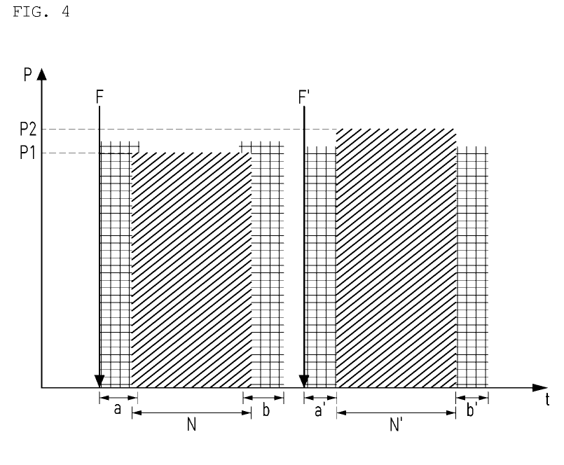

FIG. 1 is a configuration diagram of an invasive light irradiation apparatus capable of automatic output limitation according to an embodiment of the present disclosure; FIG. 2 is a schematic view of a needle part of the invasive light irradiation apparatus capable of automatic output limitation according to the embodiment of the present disclosure; FIG. 3 is a schematic view of the invasive light irradiation apparatus capable of automatic output limitation according to the embodiment of the present disclosure; and FIG. 4 is a graph showing an irradiation cycle of the invasive light irradiation apparatus capable of automatic output limitation according to the embodiment of the present disclosure.

Referring to FIGS. 1 to 4, the invasive light irradiation apparatus capable of automatic output limitation according to the present disclosure includes a main body 100 configured to enable and control operation, and a needle part 10 attachable to and detachable from the main body 100.

The main body 100 includes a power supply part 110 configured to supply power, a control part 120 configured to control operation by receiving power from the power supply part 110, a display part 130 configured to output an operational status to an operator or a patient under control of the control part 120, a light generation part 140 configured to generate light by receiving power from the power supply part 110, a light transmission part 150 configured to transmit the light generated by the light generation part 140, a sensor part 160 coupled to one side of the light transmission part 150 to detect reflected light, and a coupling part 170 configured to connect the needle part 10 to the main body 100.

The control part 120 controls the operation of the light generation part 140 according to a value input through the display part 130.

In addition, the control part 120 includes a determination part (not shown) configured to compare an intensity of light generated by the light generation part 140 and output through the light transmission part 150 with an intensity of light detected by the sensor part 160.

The light transmission part 150 includes a first light transmitter 13 coupled to one side of the light generation part 140 so as to transmit light generated by the light generation part 140, a second light transmitter 14 coupled to one side of the sensor part 160 so as to transmit reflected light to the sensor part 160 by utilizing reflective properties of light, and a first coupling member 151 configured to couple the first light transmitter 13 and the second light transmitter 14 together.

The first light transmitter 13 and the second light transmitter 14 protrude separately from a first side of the first coupling member 151, and a coupled light transmitter 20 protrudes from a second side of the first coupling member 151.

The needle part 10 includes: a second coupling member 16 detachably coupled to the coupling part 170 of the main body 100; a third light transmitter 11 connected to one side of the second coupling member 16 so as to transmit light generated from the main body 100; and an invasive device 15 coupled to one end of the third light transmitter 11 and having a pointed shape formed by being cut diagonally so as to be invasively inserted into the skin of a patient.

The coupled light transmitter 20 is coupled to the first coupling member 151 at a first side thereof, and includes a coupling terminal 20‑1, which is coupled to the second coupling member 16, at a second side thereof.

That is, light generated by the light generation part 140 of the main body 100 is transmitted to the first light transmitter 13 of the light transmission part 150, is transmitted to the coupled light transmitter 20 via the coupling of the first coupling member 151 thereto, and is transmitted to the third light transmitter 11 by coupling the coupling terminal 20-1 of the coupled light transmitter 20 to the second coupling member 16. The third light transmitter 11 has the one end coupled to the invasive device 15, which is diagonally cut and inserted into the skin of a patient, and configured to transmit light below the skin of a user through the invasive device 15.

The first light transmitter 13, the second light transmitter 14, the coupled light transmitter 20, the third light transmitter 11, and the invasive device 15 are hollow to have internal empty spaces, respectively, each of the first light transmitter 13, the second light transmitter 14, the coupled light transmitter 20, and the third light transmitter 11 being made of a flexible material that allows bending, and an optical fiber is inserted into each of the internal empty spaces. In addition, the invasive device 15 is made of a rigid steel material, and similarly, an optical fiber is inserted into the internal empty space of the invasive device 15.

That is, light generated by the light generation part 140 of the main body 100 is transmitted through the optical fibers of the first light transmitter 13 and the second light transmitter 14 of the light transmission part 150, the coupled light transmitter 20, the third light transmitter 11, and the invasive device 15.

The coupling part 170 may have one to twenty ports, but is not limited thereto. In the present disclosure, the coupling part 170 having one port is described as an example.

The coupling part 170 may further include a contact detection sensor at an end thereof, thereby sensing the coupling of the coupling part 170 to the needle part 10.

The sensor part 160 is connected to one side of the second light transmitter 14, and the second light transmitter 14 detects a portion of light that is reflected back when the light generated by the main body 100 is emitted through the needle part 10 as described above.

Due to the material characteristics of the optical fiber, all of light does not pass through the optical fiber, and a portion of the light is reflected. In this case, the sensor part 160 detects the portion of the reflected light.

In addition, when the needle part 10 is inserted into the human body, the intensity of the portion of the reflected light is lower than the intensity of light reflected when the needle part 10 is not invasively inserted into the human body, that is, when the needle part 10 is exposed to the air.

By using the above features, the determination part of the control part 120 compares an intensity of reflected light detected by the sensor part 160 with an intensity of light generated by the main body 100 and emitted to the needle part 10 so as to determine whether the needle part 10 is invasively inserted.

When the needle part 10 is invasively inserted, 0.1% to 2% of the light generated by the main body 100 is reflected, whereas when the needle part 10 is not invasively inserted, 3% to 5% of the light generated by the main body 100 is reflected.

Next, referring to FIG. 4, when the control part 120 detects an operation signal F as light energy P1, the light generation part 140 generates and outputs light, and the sensor part 160 senses reflected light for a first period of a seconds.

In this case, P1 is any one of 5 mW to 50 mW, but is not limited thereto.

In this case, the a seconds is set to any one of 0.1 seconds to 5.0 seconds, but is not limited thereto.

Next, when the needle part 10 is invasively inserted into the skin, light is continuously output for N minutes set by the control part 120. In this case, the N minutes is set within a range from 2 minutes to 30 minutes, but is not limited thereto. In this case, the sensor part 160 senses reflected light at the same time as the light is continuously output.

When the needle part 10 is invasively inserted into the skin, light is continuously output for the N minutes set by the control part 120, and, simultaneously, the sensor part 160 senses reflected light for a second period of b seconds. In this case, the b seconds is set to any one of 0.1 seconds to 5.0 seconds, but is not limited thereto. In addition, when the light is continuously output for the N minutes, the sensor part 160 simultaneously senses the reflected light for a maximum of 0.1 seconds. That is, this is intended to compare the intensity of reflected light when the needle part 10 is invasively inserted into the skin with the intensity of reflected light when the needle part 10 is removed from the skin.

Although the embodiment of the present disclosure has been described in detail above, the scope of the present disclosure is not limited thereto, and various modifications and improvements by those skilled in the art using the basic concepts of the present disclosure as defined in the following claims also fall within the scope of the present disclosure.

What is claimed is:

1. An invasive light irradiation apparatus capable of automatic output limitation, the apparatus comprising:
a main body configured to enable and control operation; and
a needle part attachable to and detachable from the main body,
wherein the main body includes:
a power supply part configured to supply power;
a control part configured to control operation by receiving power from the power supply part;
a display part configured to output an operational status to an operator or a patient under control of the control part;
a light generation part configured to generate light by receiving power from the power supply part;
a light transmission part configured to transmit light generated by the light generation part;
a sensor part coupled to one side of the light transmission part so as to detect reflected light; and
a coupling part configured to connect the needle part to the main body,
wherein the needle part includes:
a second coupling member detachably coupled to the coupling part of the main body;
a third light transmitter connected to one side of the second coupling member so as to transmit light generated from the main body; and
an invasive device coupled to one end of the third light transmitter and having a pointed shape formed by being cut diagonally so as to be inserted into skin of a patient.

2. The apparatus of claim 1, wherein the control part includes a determination part configured to compare an intensity of light generated by the light generation part and output through the light transmission part with an intensity of light detected by the sensor part.

3. The apparatus of claim 1, wherein the light transmission part includes:
a first light transmitter coupled to one side of the light generation part so as to transmit light generated by the light generation part;
a second light transmitter coupled to one side of the sensor part so as to transmit reflected light to the sensor part by utilizing reflective properties of light; and
a first coupling member configured to couple the first light transmitter and the second light transmitter together.

4. The apparatus of claim 1, wherein the coupling part has one to twenty ports.

5. The apparatus of claim 3, wherein the first light transmitter and the second light transmitter protrude separately from a first side of the first coupling member, and a coupled light transmitter protrudes from a second side of the first coupling member.

6. The apparatus of claim 5, wherein the coupled light transmitter is coupled to the first coupling member at a first side thereof and includes a coupling terminal, which is coupled to the second coupling member, at a second side thereof.

7. The apparatus of claim 3, wherein each of the first light transmitter and the second light transmitter is hollow to have an internal empty space, and is made of a flexible material that allows bending, with an optical fiber inserted into the internal hollow space.

8. The apparatus of claim 1, wherein the invasive device is hollow to have an internal empty space and is made of a rigid steel material, with an optical fiber inserted into the internal empty space of the invasive device.

9. The apparatus of claim 1, wherein light generated by the light generation part of the main body is transmitted through optical fibers of a first light transmitter and a second light transmitter of the light transmission part, a coupled light transmitter, the third light transmitter, and the invasive device.

10. The apparatus of claim 1, wherein the sensor part is connected to one side of a second light transmitter, and the second light transmitter detects a portion of light that is reflected back when the light generated by the main body is emitted through the needle part.

11. The apparatus of claim 1, wherein a determination part compares an intensity of reflected light detected by the sensor part with an intensity of light generated by the main body and emitted to the needle part so as to determine whether the needle part is invasively inserted.

12. The apparatus of claim 1, wherein when the control part detects an operation signal as light energy P1, the light generation part generates and outputs light, and the sensor part senses reflected light for a first period of a seconds,
wherein the P1 is set within a range from 5mW to 50mW, and the a seconds is set within a range from 0.1 seconds to 5.0 seconds.

13. The apparatus of claim 1, wherein when the needle part is invasively inserted into the skin, light is continuously output for N minutes set by the control part,
wherein the N minutes is set within a range from 2 minutes to 30 minutes.

14. The apparatus of claim 13, wherein when the needle part is invasively inserted into the skin, light is continuously output for the N minutes set by the control part, and, simultaneously, the sensor part senses reflected light for a second period of b seconds,
wherein the b seconds is set within a range from 0.1 seconds to 5.0 seconds.
